(19) United States
(12) Patent Application Publication
(10) Pub. No.: US 2004/0198960 A1
(43) Pub. Date: Oct. 7, 2004

(54) Human monoclonal antibodies against capsular polysaccharides of streptococcus pneumoniae

(76) Inventors: Edward N Janoff, Wayzata, MN (US); Claudine Fasching, Eden Prairie, MN (US)

Correspondence Address: MUETING, RAASCH & GEBHARDT, P.A., P.O. BOX 581415, MINNEAPOLIS, MN, 55458, US

(21) Appl. No.: 10/483,447

(22) PCT Filed: Mar. 29, 2002
(86) PCT No.: PCT/US02/10129

Related U.S. Application Data
(60) Provisional application No. 60/279,918, filed on Mar. 29, 2001.
(60) Provisional application No. 60/286,725, filed on Apr. 25, 2001.

(51) Int. Cl. C07K 16/10

(52) U.S. Cl. 530/388.4

(57) ABSTRACT

Disclosed herein are human monoclonal antibodies directed against capsular or cell wall-associated polysaccharides of Streptococcus pneumoniae and the hybridoma cell lines that secrete these antibodies. Also disclosed are therapeutic methods for treating S. pneumoniae infected individuals and prophylactic methods for treating individuals at high risk for S. pneumoniae infections by the passive administration of these human monoclonal antibodies.

### CONTINUING APPLICATION DATA

This application claims the benefit of U.S. Provisional Application Serial No. 60/279,918, filed Mar. 29, 2001, and U.S. Provisional Application Serial No. 60/286,725, filed Apr. 25, 2001, both of which are incorporated by reference herein.

### GOVERNMENT FUNDING

The present invention was made with government support under Grant No. R01-AI-48796, awarded by the National Institutes of Health. Government support was also received from the Veterans' Administration. The Government has certain rights in this invention.

### BACKGROUND

Streptococcus pneumoniae (S. pneumoniae) is the most common cause of serious lung infections in children and adults in the US and worldwide. Respiratory infection with the bacterium S. pneumoniae leads to an estimated 500,000 cases of pneumonia and 47,000 deaths annually. Those persons at highest risk of bacteremic pneumococcal infection are infants under two years of age, individuals with compromised immune systems and the elderly. In these populations, S. pneumoniae is the leading cause of bacterial pneumonia and meningitis. S. pneumoniae is also the leading cause of invasive bacterial respiratory disease in both adults and children with HIV infection and produces hematogenous infection in these patients (Connor et al. Current Topics in AIDS 1987;1:185-209 and Janoff et al. Ann. Intern. Med. 1992;117(4):314-324). Moreover, S. pneumoniae is the major bacterial cause of ear infections in children of all ages. S. pneumoniae may colonize the nasopharyngeal mucosa without sequelae but also commonly causes serious invasive clinical syndromes, including pneumonia, bacteremia, and meningitis in children and adults.

A polysaccharide capsule surrounds the bacteria and prevents it from ingestion by phagocytic cells. Capsular polysaccharides (PPS) of S. pneumoniae are a prominent target of protective antibodies. Although PPS were the first non-protein antigens described, the molecular and functional characteristics of antibodies to PPS are not well characterized. Natural infection and immunization elicit capsule-specific polymeric IgA (pIgA) responses in blood and at mucosal sites. Both children and the elderly share defects in the synthesis of protective antibodies to pneumococcal capsular polysaccharide after either bacterial colonization, local or systemic infection, or vaccination with purified polysaccharides.

Individuals who demonstrate the greatest risk for severe infection are not able to make antibodies to the current capsular polysaccharide vaccines. As a result, conjugate vaccines consisting of pneumococcal capsular polysaccharides coupled to protein carriers or adjuvants in an attempt to boost the antibody response, are currently in clinical trial. However, there are other potential problems with conjugate vaccines currently in clinical trials. For example, pneumococcal serotypes that are most prevalent in the United States are different from the serotypes that are most common in places such as Israel, Western Europe, South Africa, or Scandinavia. Therefore, vaccines that may be useful in one geographic locale may not be useful in another. The potential need to modify currently available capsular polysaccharide vaccines or to develop protein conjugates for capsular vaccines to suit geographic serotype variability entails prohibitive financial and technical complications.

In patients who cannot make protective antibodies to the type-specific polysaccharide capsule, other therapeutic approaches are needed. There currently exists a strong need for methods and therapies to limit S. pneumoniae infections. Moreover, the emergence of penicillin and cephalosporin-resistant pneumococci on a worldwide basis increases the urgency of this need. See e.g. Schwartz, New Engl J Med 348(10):722, 2002 and Baquero et al. J. Antimicrob. Chemother. 28S:31-8, 1991.

Previous findings have shown a significant IgA response to pneumococcal capsular polysaccharides after immunization and natural infection. Indeed, IgA comprises an appreciable proportion of capsule-specific IgA in immune serum. The role of IgA in the control of invasive mucosal pathogens such as S. pneumoniae is not completely understood. Human pneumococcal capsular polysaccharide-specific IgA initiated dose-dependent killing of S. pneumoniae with complement and phagocytes has been demonstrated. Janoff et al., J Clin Invest 104:1139-1147, 1999. In these demonstrations, the majority of specific IgA in serum was of the polymeric form (pIgA), and the efficiency of pIgA-initiated killing exceeded that of monomeric IgA initiated killing. In the absence of complement, specific IgA induced minimal bacterial adherence, uptake, and killing. Killing of S. pneumoniae by resting phagocytes with immune IgA required complement, predominantly via the C2-independent alternative pathway, which requires factor B, but not calcium. Both S. pneumoniae-bound IgA and complement were involved, as demonstrated by a 50% decrease in killing with blocking of Fc α receptor (CD89) and CR1/CR3 (CD35/CD11b). However, IgA-mediated killing by phagocytes could be reproduced in the absence of opsonic complement by pre-activating phagocytes with the inflammatory products C5a and TNF-α. Thus, S. pneumoniae capsule-specific IgA may show distinct roles in effecting clearance of S. pneumoniae in the presence or absence of inflammation. These data suggest mechanisms whereby pIgA may serve to control pneumococcal infections locally and upon the pathogen's entry into the bloodstream.

Recent work has also shown that up to a third of serum antibodies that are reactive with the polysaccharide capsule of an invasive mucosal Gram-positive organism, S. pneumoniae, are of the IgA class. The majority of pathogen-specific IgA in serum is in the polymeric form and remains polymeric long after immunization or infection, even though about 90% of the total IgA in serum is in the monomeric form. Janoff et al., J Clin Invest 104:1139-1147, 1999.

### SUMMARY OF THE INVENTION

The present invention includes human monoclonal antibodies that specifically bind to a capsular or cell wall-associated polysaccharide antigen of Streptococcus pneumoniae. These antibodies include the human monoclonal antibodies 1A01, 2A01, 2A02, 2A03, 2G01, 2A04, 3A01, 3G01, 4G01, 7A01, 8A01, 8A04, 8A02, 8G01, 9G01, 33G01, 18CA01, 22FG01, 6BG01, 6BA01 and CPSM01; and antigen-binding fragments thereof. These antibodies also include the human monoclonal antibodies secreted by the hybridoma cell lines Z727 1D8 1C7 2B8; BBK040 1F2 1G7 2G8; Z531 3D7 5A9 1A9; Z531 4B6 3F5 1E2; Z727 1C11 1E12 3B10; Z727 1D11 1D11 1F6; Z531 1F9 3H9 1B7; Z727 2G6 1B5 3B7; Z727 2B5; Z727 1G8 1B10 3E1; 1A8 1A3/1F7/1H59; BBK038 2F4 F11 1B6; BBK040 1D9 2B5; BBK040 4E4 2D1 1A5; BBK038 1B8 E2 1F6; Z727 1G8 1B10 1E12/3D5; Z727 1C11 1F7 2A8; Z727 1G8 1B3 1B11/1F9; Z727 1D11 1H6; BBK040 4E4 1A2; Z727 1D11 1E7 2E1; and BBK041 2G5 1G2. The antigen-binding fragments of the present invention may include scFv, Fv, Fab′, Fab, diabody, linear antibody or F(ab′)₂ fragments.

Also included in the present invention are antibodies or antigen-binding fragment that specifically bind to a capsular or cell wall-associated polysaccharide antigen of Streptococcus pneumoniae, the antibodies or antigen-binding fragments having a VH region amino acid sequence encoded by a polynucleotide sequence selected from SEQ ID NO:14-26. Also included in the invention are antibodies or antigen-binding fragments that specifically bind to a capsular or cell wall-associated polysaccharide antigen of Streptococcus pneumoniae, the antibodies or antigen-binding fragments having a CDR3 amino acid sequence selected from SEQ ID NO:1-13.

The invention also includes the hybridoma cell lines Z727 1D8 1C7 2B8; BBK040 1F2 1G7 2G8; Z531 3D7 5A9 1A9; Z531 4B6 3F5 1E2; Z727 1C11 1E12 3B10; Z727 1D11 1D11 1F6; Z531 1F9 3H9 1B7; Z727 2G6 1B5 3B7; Z727 2B5; Z727 1G8 1B10 3E1; 1A8 1A3/1F7/1H59; BBK038 2F4 F11 1B6; BBK040 1D9 2B5; BBK040 4E4 2D1 1A5; BBK038 1B8 E2 1F6; Z727 1G8 1B10 1E12/3D5; Z727 1C11 1F7 2A8; Z727 1G8 1B3 1B11/1F9; Z727 1D11 1H6; BBK040 4E4 1A2; Z727 1D11 1E7 2E1; and BBK041 2G5 1G2.

The invention also includes pharmaceutical compositions containing at least one of the described antibodies that bind to a capsular or cell wall-associated polysaccharide antigen of Streptococcus pneumoniae. This pharmaceutical composition may also include a pharmaceutically acceptable carrier. The pharmaceutical composition of the present invention may include a cocktail of at least one of the human monoclonal antibodies that specifically bind to a capsular or cell wall-associated polysaccharide antigen of Streptococcus pneumoniae, or antigen-binding fragments thereof.

Also included in the present invention are methods for treating individuals with a Streptococcus pneumoniae infection by passively administering a therapeutically effective amount of a pharmaceutical composition of at least one human monoclonal antibody that specifically binds to a capsular or cell wall-associated polysaccharide antigen of Streptococcus pneumoniae. This individual may be a human patient. This human patient may suffer from pneumococcal pneumonia, meningitis, otis media, sinusitis, sickle cell anemia, hypogammaglobulinemia, asplenia or bacteremia. This patient may have an impaired ability to produce antibodies. The patient may be under two years of age or experience recurrent meningitis because of a skull fracture or other structural defect.

Also included in the present invention are methods for preventing a

Streptococcus pneumoniae infection in an individual by passively administering a prophylactically effective amount of a pharmaceutical composition of at least one human monoclonal antibody that specifically binds to a capsular or cell wall-associated polysaccharide antigen of Streptococcus pneumoniae. This individual may suffer from HIV/AIDS, functional or surgical asplenia, chronic lymphocytic leukemia (CLL), multiple myeloma (MM), hypogammaglobulinemia or sickle cell anemia. The patient may be under two years of age or experience recurrent meningitis because of a skull fracture or other structural defect.

The invention also includes a method for treating an individual with a Streptococcus pneumoniae infection by a) selecting at least one human monoclonal antibody that specifically binds to a capsular or cell wall-associated polysaccharide antigen of Streptococcus pneumoniae, utilizes a heavy-chain immunoglobulin gene from the VH3 gene family, comprises a shortened CDR3 region of 8-12 amino acids, demonstrates an increased mutation rate of 6.5±0.9% and demonstrates a decreased proportion of solvent-exposed amino acids relative to an antibody specific for a protein antigen and b) passively administering a therapeutically effective amount of this human monoclonal antibody to the individual. Additionally, the present invention includes a method for preventing a Streptococcus pneumoniae infection in an individual by a) selecting at least one human monoclonal antibody that specifically binds to a capsular or cell wall-associated polysaccharide antigen of Streptococcus pneumoniae, utilizes a heavy-chain immunoglobulin gene from the VH3 gene family, comprises a shortened CDR3 region of 8-12 amino acids, demonstrates an increased mutation rate of 6.5±0.9% and demonstrates a decreased proportion of solvent-exposed amino acids relative to an antibody specific for a protein antigen and b) passively administering a prophylactically effective amount of this human monoclonal antibody to the individual.

The present invention also includes methods of detecting Streptococcus pneumoniae by contacting a biological sample with one of the human monoclonal antibodies that specifically bind to a capsular or cell wall-associated polysaccharide antigen of Streptococcus pneumoniae.

Also included in the present invention are anti-idiotypic antibodies that bind to the human monoclonal antibodies that bind to a capsular or cell wall-associated polysaccharide antigen of Streptococcus pneumoniae.

### BRIEF DESCRIPTION OF THE FIGURES

FIG. 1 is a chart characterizing 21 S. pneumoniae-specific human monoclonal antibodies representative of the present invention.

FIG. 2 is a graphical representation of V_{H} gene expression in pathogen-specific antibodies. Ninety-two human monoclonal antibodies were characterized; twenty-two were specific for S. pneumoniae capsule (PPS), thirty-one were specific for H. influenzae b capsule (PRP), and thirty-nine were specific for various protein pathogens (HepB, RSV, CMV, parvovirus B19, rabies, OMT and TT).

FIG. 3 represents the mutation rate in the V_{H} CDR and FR regions of ninety-two pathogen-specific human monoclonal antibodies.

FIG. 4 is a representation of CDR3 length in amino acids grouped by antigen specificity for ninety-two pathogen-specific human monoclonal antibodies.

FIG. 5 is a representation of the pI of CDR3 grouped by antigen specificity for the ninety-two pathogen-specific human monoclonal antibodies.

FIG. 6 is composite listing of the amino acid composition of V_{H} CDR3 regions of antibodies with specificity for PPS, PRP and protein pathogens.

FIG. 7 represents the virulence of S. pneumoniae Type 3 in mice. LD50=50% lethal dose i.p. in CFU.

FIG. 8 shows the survival rate (%) of mice protected with the IgA human monoclonal clone 2A02 against intratracheal challenge with S. pneumoniae type 2.

FIG. 9 shows the polynucleotide sequences of the V_{H} region of S. pneumoniae-specifc monoclonal antibodies.

### DETAILED DESCRIPTION OF PREFERRED EMBODIMENTS OF THE INVENTION

The present invention is illustrated by the following examples. It is to be understood that the particular examples, materials, amounts, and procedures are to be interpreted broadly in accordance with the scope and spirit of the invention as set forth herein.

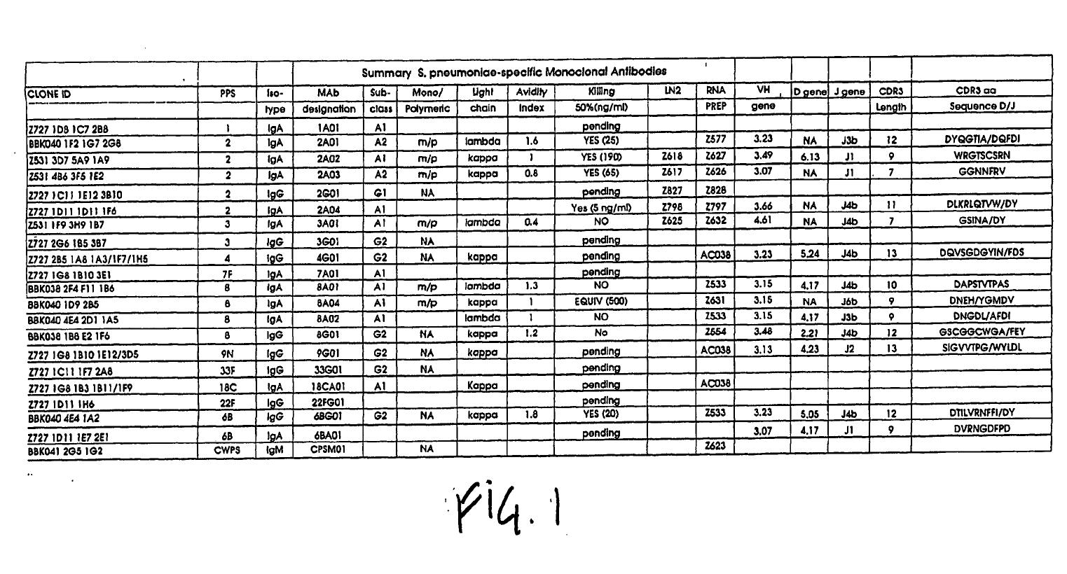

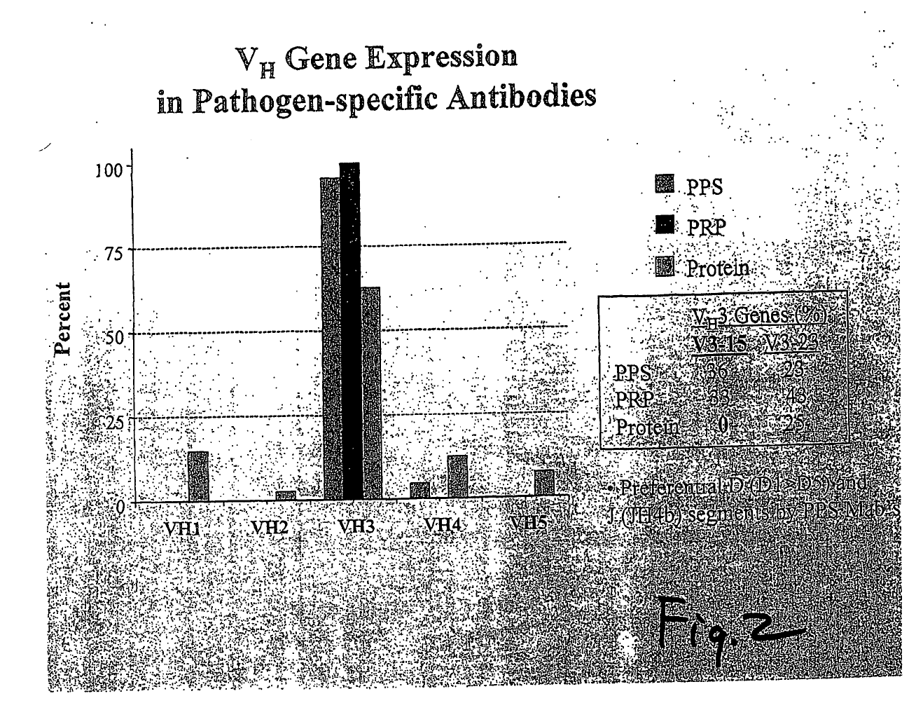

This invention relates to human monoclonal antibodies that specifically bind to capsular polysaccharides antigens of S. pneumoniae. As shown in FIG. 1, twenty-one human monoclonal antibodies directed against capsular polysaccharides of S. pneumoniae have been developed. Since the pioneering work of Kohler and Milstein, the production of mouse monoclonal antibodies has become routine. However, as discussed in U.S. Pat. No. 6,197,582, the application of xenogenic monoclonal antibodies for in vivo diagnostics and therapy in humans is often associated with undesirable effects. For example, the administration of murine antibodies to humans can cause a dangerous life threatening immunologic reaction, a human anti-mouse immunoglobulin response. Thus, human monoclonal antibodies have great potential as tools in imaging and therapeutic treatment. Many different procedures are known for the production of hybridoma cell lines that secrete a human monoclonal antibodies. The use of Epstein-Barr virus (EBV) has proved to be quite efficient for human lymphocyte immortalization, but has certain limitations such as low antibody secretion rate, poor clonogenicity of antibody-secreting lines and chromosomal instability requiring frequent subcloning. In another method of generating human monoclonal antibodies, human B lymphocytes are isolated, activated with Epstein-Barr virus (EBV), and fused to mouse-human heteromyeloma cell lines by electrofusion.

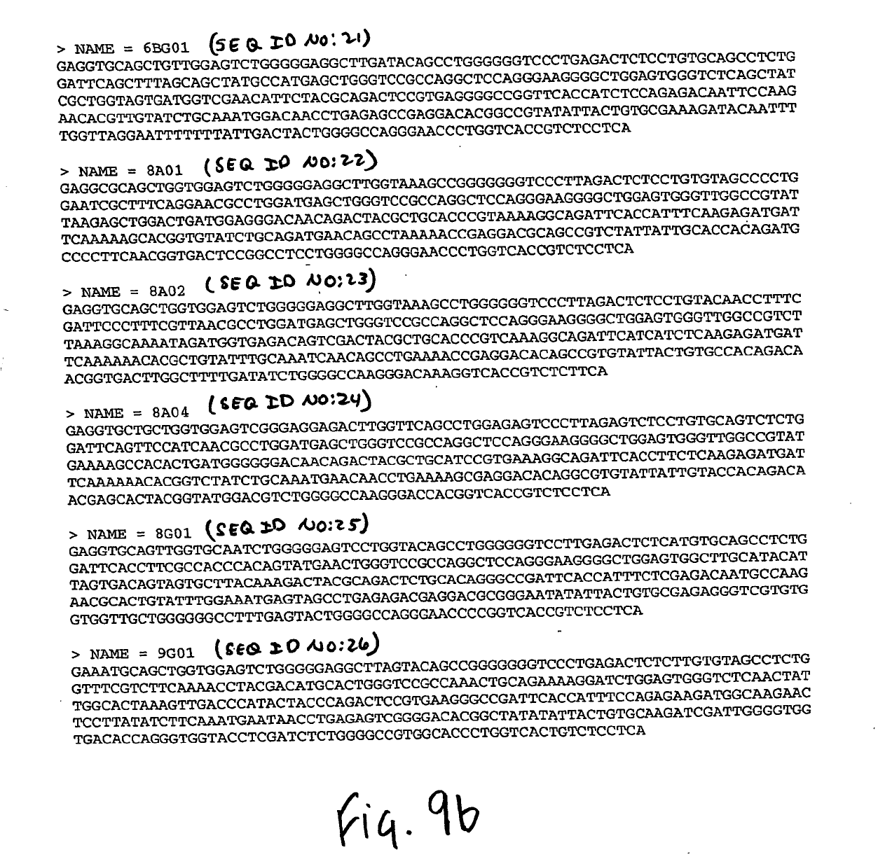

For the present invention, hybridoma cell lines that secrete S. pneumoniae-specific human monoclonal antibodies were generated by fusing B cells of immunized healthy adults with K6H6/B5 mouse-human heteromyeloma cells using the method set forth in Example 2. As shown in FIG. 1, twenty-one human monoclonal antibodies directed against capsular polysaccharides of S. pneumoniae have been developed. These twenty-one hybridoma cells lines (identified by clone ID number) are as follows: Z727 1D8 1C7 2B8; BBK040 1F2 1G7 2G8; Z531 3D7 5A9 1A9; Z531 4B6 3F5 1E2; Z727 1C11 1E12 3B10; Z727 1D11 1D11 1F6; Z531 1F9 3H9 1B7; Z727 2G6 1B5 3B7; Z727 2B5; Z727 1G8 1B10 3E1; 1A8 1A3/1F7/1H59; BBK038 2F4 F11 1B6; BBK040 1D9 2B5; BBK040 4E4 2D1 1A5; BBK038 1B8 E2 1F6; Z727 1G8 1B10 1E12/3D5; Z727 1C11 1F7 2A8; Z727 1G8 1B3 1B11/1F9; Z727 1D11 1H6; BBK040 4E4 1A2; Z727 1D11 1E7 2E1; BBK041 2G5 1G2. The monoclonal antibody secreted by each of these clones is as follows: 1A01, 2A01, 2A02, 2A03, 2G01, 2A04, 3A01, 3G01, 4G01, 7A01, 8A01, 8A04, 8A02, 8G01, 9G01, 33G01, 18CA01, 22FG01, 6BG01, 6BA01, and CPSM01, respectively. FIG. 1 summarizes the physiochemical and functional properties of these monoclonal antibodies, as follows. The identification of the PPS serotype bound (1, 2, 3, 4, 6B, 7F, 8, 9N, 12F, 14, 18C, 19A, 19F, 22F, 23F or 33F), was determined as outlined in Example 4. The isotype (IgA or IgG) and subclass (IgA1, IgA2, IgG1 or IgG2) was determined for each antibody as outlined in Example 6(a). The determination of whether the antibody is monomeric or polymeric was as outlined in Example 6(b). This determination of whether the antibody is monomeric or polymeric was not performed on IgG antibodies, indicated by a NA in FIG. 1. The determination of whether a lambda or kappa light chain was used was as outlined in Example 6(a). The avidity index was determined as outlined in Example 6(c). Killing ability was determined as outlined in Example 6(c). Using the methods outlined in Example 6(d), the complete nucleotide sequence of the VH region was determined. This sequence information is presented in FIG. 9. From this sequence information, V, D and J gene segment usage to form the VH region, the length of the CDR3 region (in amino acids) and the amino acid sequence of the D/J region within the CDR3 was determined. The amino acid sequences of the CDR3 region for various S. pneumoniae-specific human monoclonal antibodies is presented in Table 1.

TABLE 1
Amino acid sequence of the CDR3 D/J region

| Antibody | CDR3 D/J sequence | SEQ ID NO: |
|---|---|---|
| 2A01 | DYQGTIADQFDI | SEQ ID NO:1 |
| 2A02 | WRGTSCSRN | SEQ ID NO:2 |
| 2A03 | GGNNFRV | SEQ ID NO:3 |
| 2A04 | DLKRLQTVWDY | SEQ ID NO:4 |
| 3A01 | GSINADY | SEQ ID NO:5 |
| 4G01 | DQVSGDGYINFDS | SEQ ID NO:6 |
| 8A01 | DAPSTVTPAS | SEQ ID NO:7 |
| 8A04 | DNEHYGMDV | SEQ ID NO:8 |
| 8A02 | DNGDLAFDI | SEQ ID NO:9 |
| 8G01 | GSCGGCWGAFEY | SEQ ID NO:10 |
| 9G01 | SIGVVTPGWYLDL | SEQ ID NO:11 |
| 6BG01 | DTILVRNFFIDY | SEQ ID NO:12 |
| 6BA01 | DVRNGDFPD | SEQ ID NO:13 |

Certain hybridomas produce human monoclonal antibodies specific for S. pneumoniae that according to aspects of the present invention that are described and referred to herein will be deposited with the American Type Culture Collection (ATCC) located at P.O. Box 1549, Manassas, Va., USA, 20108, pursuant to the Budapest Treaty. Samples of the deposited hybridomas will become available to the public upon grant of a patent based upon this United States patent application. The invention described and claimed herein is not to be limited in scope by the hybridomas deposited, since the deposited embodiment is intended only as an illustration of the invention. Any equivalent or similar hybridomas that produce similar or equivalent antibodies as described in this application are within the scope of the invention.

### Deposit Summary

| | | |
|---|---|---|
| Hybridomas | ATCC Designation | Date Deposited |

| | |
|---|---|
| Z727 1D8 1C7 2B8 | (1A01) |
| BBK040 1F2 1G7 2G8 | (2A01) |
| Z531 3D7 5A9 1A9 | (2A02) |
| Z531 4B6 3F5 1E2 | (2A03) |
| Z727 1C11 1E12 3B10 | (2G01) |
| Z727 1D11 1D11 1F6 | (2A04) |
| Z531 1F9 3H9 1B7 | (3A01) |
| Z727 2G6 1B5 3B7 | (3G01) |
| Z727 2B5 1A8 1A3/1F7/1H59 | (4G01) |
| Z727 1G8 1B10 3E1 | (7A01) |
| BBK038 2F4 F11 1B6 | (8A01) |
| BBK040 1D9 2B5 | (8A04) |
| BBK040 4E4 2D1 1A5 | (8A02) |
| BBK038 1B8 E2 1F6 | (8G01) |
| Z727 1G8 1B10 1E12/3D5 | (9G01) |
| Z727 1C11 1F7 2A8 | (33G01) |
| Z727 1G8 1B3 1B11/1F9 | (18CA01) |
| Z727 1D11 1H6 | (22FG01) |
| BBK040 4E4 1A2 | (6BG01) |
| Z727 1D1 1E7 2E1 | (6BA01) |
| BBK041 2G5 1G2 | (CPSM01) |

The terms “antibody” and “immunoglobulin,” as used herein, refer broadly to any immunological binding agent. Depending on the type of constant domain in the heavy chain, antibodies are assigned to one of five major classes: IgA, IgD, IgE, IgG, and IgM. The heavy-chain constant domains that correspond to the different classes of immunoglobulins are termed α, δ, ε, γ and μ, respectively. Several of these are further divided into subclasses or isotypes, such as IgG1, IgG2, IgG3, IgG4, and the like. The light chains of mammalian antibodies are assigned to one of two clearly distinct types: kappa (κ) and lambda (λ), based on the amino acid sequences of their constant domains. The subunit structures and three-dimensional configurations of these different classes of immunoglobulins are well known.

As will be understood by those in the art, the immunological binding reagents encompassed by the term “antibody” extend to all antibodies from all species, and antigen binding fragments thereof, including dimeric, trimeric and multimeric antibodies; bispecific antibodies; chimeric antibodies; human and humanized antibodies; recombinant and engineered antibodies, and fragments thereof. The term “antibody” as used in this invention includes intact immunoglobulin molecules as well as antigen-binding fragments thereof which are capable of binding the same epitopic determinant. As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics. Irrespective of the source of the original, intact antibody, antibody multimers, or any one of a variety of functional, antigen-binding regions of the antibody are included in the present invention. Exemplary functional regions include diabodies, linear antibodies and scFv, Fv, Fab′, Fab, F(ab′)₂ fragments of the anti-S. pneumoniae antibodies. Techniques for preparing such constructs are well known to those in the art.

Antibody fragments can be obtained by proteolysis of the whole immunoglobulin by the non-specific thiol protease, papain. Papain digestion yields two identical antigen-binding fragments, termed “Fab fragments,” each with a single antigen-binding site, and a residual “Fc fragment.” The usual procedure for preparation of F(ab′)₂ fragments from IgG of human origin is limited proteolysis by the enzyme pepsin. Pepsin treatment of intact antibodies yields an F(ab′)₂ fragment that has two antigen-combining sites and is still capable of cross-linking antigen. An Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. F(ab′)₂ antibody fragments were originally produced as pairs of Fab′ fragments that have hinge cysteines between them. Other chemical couplings of antibody fragments are also known.

An “Fv” fragment is the minimum antibody fragment that contains a complete antigen-recognition and binding site. This region consists of a dimer of one heavy chain and one light chain variable domain in tight, con-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. “Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains that enables the sFv to form the desired structure for antigen binding. The following references provide instruction for preparation and use of functional, antigen-binding regions of antibodies, including scFv, Fv, Fab′, Fab and F(ab′)₂ fragments: U.S. Pat. Nos. 6,342,219; 5,855,866; 5,965,132; 6,051,230; 6,004,555; and 5,877,289. WO 98/45331 describes and teaches the preparation of variable, hypervariable and complementarity determining (CDR) regions of antibodies.

“Diabodies” are small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described in EP 404,097 and WO 93/11161. “Linear antibodies,” which can be bispecific or monospecific, comprise a pair of tandem Fd segments (V_{H}-C_{H1}-V_{H}-C_{H1}) that form a pair of antigen binding regions. Zapata et al., Protein Eng. 8(10):1057-62, 1995, describes the production of such linear antibodies.

With the provision of structural and functional information for a biological molecule a range of equivalent, or even improved molecules can be generated. This applies to the immunoglobulins of the present invention. Although antigen-binding and other functional properties of an antibody must be conserved, there is an extremely high degree of skill in the art in making equivalent and even improved antibodies once a reference antibody has been provided. Such technical skill can, in light of the sequences and information provided herein, be applied to the production of further antibodies that have like, improved or otherwise desirable characteristics.

For equivalent antibodies, certain amino acids may substituted for other amino acids in the antibody constant or variable domain framework regions without appreciable loss of interactive binding capacity. Other types of variants are antibodies with improved biological properties relative to the parent antibody from which they are generated. Such variants, or second generation compounds, are typically substitutional variants involving one or more substituted hypervariable region residues of a parent antibody. A convenient way for generating such substitutional variants is affinity maturation using phage display. Alternatively, or in addition, it is contemplated that the crystal structure of the antigen-antibody complex be delineated and analyzed to identify contact points between the antibody and antigen. Such contact residues and neighboring residues are candidates for substitution. Once such variants are generated, the panel of variants is subjected to screening, as described herein, and antibodies with analogues but different or even superior properties in one or more relevant assays are selected for further development.

Human monoclonal antibodies of this invention can also be produced by altering the nucleotide sequence of a polynucleotide sequence that encodes a heavy or light chain of a monoclonal antibody of this invention. For example, by site directed mutagenesis, one can alter the nucleotide sequence of an expression vector and thereby introduce changes in the resulting expressed amino acid residue sequence. Site-directed and random mutagenesis methods are well known in the polynucleotide arts, and are not to be construed as limiting as methods for altering the nucleotide sequence of a subject polynucleotide.

The terms “bind to about, substantially or essentially the same” or “bind the same epitope as” a given monoclonal antibody mean that an antibody “cross-reacts” with the given monoclonal antibody. “Cross-reactive antibodies” are those that recognize, bind to or have immunospecificity for substantially or essentially the same epitope or “epitopic site” as a given monoclonal antibody such that are able to effectively compete with the monoclonal antibody for binding to the antigen.

The human monoclonal antibodies of the present invention can also be used immunotherapeutically. The term “immunotherapeutically” or “immunotherapy” as used herein in conjunction with the monoclonal antibodies of the invention denotes both prophylactic as well as therapeutic administration. Thus, the monoclonal antibodies can be administered to patients in order to lessen the likelihood and/or severity of S. pneumoniae infection, or administered to patients already evidencing active S. pneumoniae infection.

The antibodies of the invention can be administered parenterally, by injection or by gradual infusion over time. The antibodies can be administered intravenously, intraperitoneally, intramuscularly, subcutaneously, or intracavity. They may be administered via the intravenous or intramuscular route. The number of treatments required to control a patient's disease will vary from individual to individual, depending upon the severity and stage of the illness and the individual characteristics of each patient being treated. The total dose required for each treatment may be administered by multiple doses or in a single dose. The human monoclonal antibodies may be administered alone or in conjunction with other agents. The dosage ranges for the immunotherapeutic administration of the monoclonal antibodies of the invention are those large enough to produce the desired effect in which the symptoms are ameliorated or the likelihood of infection is decreased. The dosage should not be so large as to cause adverse side effects, such as hyperviscosity syndromes, pulmonary edema, conjestive heart failure, and the like. Generally, the dosage will vary with the age, condition, sex and extent of the disease in the patient and can be determined by one of skill in the art. The dosage can be adjusted by the individual physician in the event of any complication. Dosage can vary from about 0.01 mg/kg to about 300 mg/kg, preferably from about 0.1 mg/kg to about 200 mg/kg, most preferably from about 0.2 mg/kg to about 20 mg/kg, in one or more dose administrations daily, for one or several days.

A formulation containing the monoclonal antibodies of the invention may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Antibodies of the present invention may be administered by passive immunization to a wide range of individuals for either therapeutic and prophylactic treatment. For example, individuals presenting with an active S. pneumoniae infection may benefit from the administration of the antibodies. Such individuals include persons of all ages with recurrent pneumonia, sinusitis, and otitis media; patients with genetic defects that impair their ability to make antibodies (hypogammaglobulinemia); patients with acquired antibody defects (such as those with chronic lymphocytic leukemia (CLL) and multiple myeloma); patients with HIV/AIDS; individuals with sickle cell disease; young children under the age of two years old; the elderly; pregnant women; persons with other selective antibody defects (e.g., IgA deficiency with IgG subclass deficiency); persons unable to respond to polysaccharides; persons with certain types of skull fractures who experience recurrent meningitis; persons with severe acute pneumococcal infection (e.g., bacteremia or meningitis); individuals who experience extremely high rates of pneumococcal infections and respond poorly to active immunization with vaccine; and individuals with drug-resistant S. pneumoniae infections.

Antibodies of the present invention may also be administered prophylactically, to prevent S. pneumoniae infection, to a wide range of individuals. For example, persons who may benefit from the administration of these antibodies include individuals with functional or surgical asplenia; patients with genetic defects that impair their ability to make antibodies (hypogammaglobulinemia); patients with acquired antibody defects (such as those with chronic lymphocytic leukemia (CLL) and multiple myeloma); patients with HIV/AIDS; individuals with sickle cell disease; persons with other selective antibody defects (e.g., IgA deficiency with IgG subclass deficiency); persons unable to respond to polysaccharides; persons with certain types of skull fractures or other structural defects who experience recurrent meningitis and individuals who experience extremely high rates of pneumococcal infections and respond poorly to active immunization with vaccine.

Commercially available immunoglobulin, which contains a high concentration of S. pneumoniae-specific immunoglobulins, is routinely administered intravenously to patients with hypogammaglobulinemia to decrease infections with encapsulated pathogens, such as S. pneumoniae. See “Cooperative Group for the Study of Immunoglobulin in Chronic Lymphocytis Leukemia,” New Engl J Med 319:902-907, 1988; and “Hypogammaglobulinaemia in the United Kingdom,” Lancet 1:163-168, 1969. The intravenous administration of commercial immunoglobulin to symptomatic HIV-1 infected children with low CD4+ T cell counts has been shown to result in a significant decrease in the rate of serious bacterial infections, including S. pneumoniae infections. See Mofenson et al., J.A.M.A. 268:483-488, 1992; Spector et al., N. Engl. J. Med. 331:1181-1187, 1994; and “National Institutes of Child Health and Human Development Intravenous Immunoglobulin Study Group,” N Engl J Med 325:73-80, 1991.

The present invention includes the passive administration of S. pneumoniae-specific human monoclonal antibodies or antigen-binding fragments of these antibodies, singly or as a cocktail to an individual for the treatment or prophylaxis of S. pneumoniae infections. A cocktail contains at least one S. pneumoniae-specific antibodies. A cocktail may contain 1, 2, 3, 4, 5, 6, 7, 8, 9, 10, 11, 12, 13, 14, 15, 16, 17, 18, 19, 20, 21, 25, or more different S. pneumoniae-specific antibodies. The cocktail may contain at least one antibody selected from those listed on FIG. 1. The cocktail may also contain S. pneumoniae-specific antibodies other than those listed in FIG. 1. A cocktail may contain antibodies with a specificity for other pathogens. A cocktail may contain other therapeutic agents, such as, but not limited to, antibiotics, anti-viral agents and anti-inflammatory agents. A cocktail is a composition comprising isolated antibodies, each antibody of the composition with an identified antigen-binding specificity. A cocktail is not an antiserum, it does not contain antibodies with unidentified antigen-binding specificities.

A composition is said to be “pharmaceutically acceptable” if its administration can be tolerated by a recipient patient. Such an agent is said to be administered in a “therapeutically effective amount” or “prophylactically effective amount” if the amount administered is physiologically significant. An agent is physiologically significant if its presence results in a detectable change in the physiology of a recipient patient. Accordingly, the effective amount will vary with the subject being treated, as well as the condition to be treated. For the purposes of this invention, the methods of administration are to include, but are not limited to, administration cutaneously, subcutaneously, intravenously, parenterally, orally, topically, or by aerosol.

As used herein, the term “pharmaceutically acceptable carrier” encompasses any of the standard pharmaceutically accepted carriers, such as phosphate buffered saline solution, water, emulsions such as an oil/water emulsion or a triglyceride emulsion, various types of wetting agents, tablets, coated tablets and capsules. An example of an acceptable triglyceride emulsion useful in intravenous and intraperitoneal administration of the compounds is the triglyceride emulsion commercially known as Intralipid.RTM. Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients. Attachment of polyethylene glycol (PEG) to compounds is particularly useful because PEG has very low toxicity in mammals. For example, a PEG adduct of adenosine deaminase was approved in the United States for use in humans for the treatment of severe combined immunodeficiency syndrome. The carrier includes a microencapsulation device so +as to reduce or prevent an host immune response against the compound or against cells which may produce the compound. The compound of the present invention may also be delivered microencapsulated in a membrane, such as a liposome.

The human monoclonal antibodies of the present invention may be employed as standards in conventional diagnostic assays. The monoclonal antibodies of the invention are also suited for in vitro use, for example, in immunoassays in which they can be utilized in liquid phase or bound to a solid phase carrier. In addition, the monoclonal antibodies in these immunoassays can be detectably labeled in various ways. Examples of types of immunoassays which can utilize monoclonal antibodies of the invention are competitive and non-competitive immunoassays in either a direct or indirect format. Examples of such immunoassays are the radioimmunoassay (RIA) and the sandwich (immunometric) assay. Detection of the antigens using the monoclonal antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation. For purposes of the invention, S. pneumoniae may be detected by the monoclonal antibodies of the invention when present in biological fluids and tissues. Any sample potentially containing S. pneumoniae may be used. A sample can be a liquid such as urine, saliva, cerebrospinal fluid, blood, serum, urine and the like, or a solid or semi-solid such as a needle aspiration or a solid tissue such as those commonly used in histological diagnosis.

This invention also provides kits comprising at least one human monoclonal antibody specific to S. pneumoniae for use in the present treatment methods. Such kits will generally contain, in suitable container means, a pharmaceutically acceptable formulation of at least one human monoclonal antibody specific to S. pneumoniae. The kits may also contain other pharmaceutically acceptable formulations, either for diagnosis/imaging or combined therapy. For example, such kits may contain any one or more of a range of chemotherapeutic, such as an antibiotic. Such kits may also be used as standard in diagnostic immunoassays.

The kits may have a single container (container means) that contains the at least one human monoclonal antibody specific to S. pneumoniae, with or without any additional components, or they may have distinct containers for each desired agent. Where combined therapeutics are provided, a single solution may be pre-mixed, either in a molar equivalent combination, or with one component in excess of the other. Alternatively, each of the at least one human monoclonal antibody specific to S. pneumoniae components of the kit may be maintained separately within distinct containers prior to administration to a patient.

When the components of the kit are provided in one or more liquid solutions, the liquid solution is preferably an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

The containers of the kit will generally include at least one vial, test tube, flask, bottle, syringe or other container means, into which the at least one human monoclonal antibody specific to S. pneumoniae, and any other desired agent, may be placed and, preferably, suitably aliquoted. Where separate components are included, the kit will also generally contain a second vial or other container into which these are placed, enabling the administration of separated designed doses. The kits may also comprise a second/third container means for containing a sterile, pharmaceutically acceptable buffer or other diluent.

The kits may also contain a means by which to administer the at least one human monoclonal antibody specific to S. pneumoniae to an animal or patient, e.g., one or more needles or syringes, or even an eye dropper, pipette, or other such like apparatus, from which the formulation may be injected into the animal or applied to a diseased area of the body. The kits of the present invention will also typically include a means for containing the vials, or such like, and other component, in close confinement for commercial sale, such as, e.g., injection or blow-molded plastic containers into which the desired vials and other apparatus are placed and retained.

The present invention includes the administration of anti-idiotype antibodies (Ab2s) mimicking S. pneumoniae polysaccharide antigens for the treatment and prevention of streptococcal infections. Ab2s are antibodies directed against the variable regions of conventional antibodies (Ab1). Certain Ab2s (termed “Ab2B,” “anti-idiotype” or “internal-image” antibodies) can mimic the three-dimensional structure of the nominal antigen, and thus Ab2 and antigen can bind with the same regions of the Ab1-combining site. Jerne et al., EMBO J. 1: 243 (1982); Losman et al., Int. J. Cancer 46: 310 (1990); Losman et al., Proc. Nat'l Acad. Sci. USA 88: 3421 (1991); Losman et al., Int. J. Cancer 56: 580 (1994). Individuals immunized with Ab2B can develop anti-anti-antibodies (Ab3), some of which can bind the nominal antigen.

The antigen mimicry properties of anti-idiotype antibodies have led to the use of Ab2B as surrogate antigens (or idiotype vaccines), when the nominal antigen is not readily available or when the host is tolerant to the nominal antigen. In experimental systems, immunization with Ab2B mimicking certain tumor-associated antigens creates specific immunity to the tumor antigen and protect against subsequent tumor growth. See, for example, Nepom et al., Proc. Nat'l Acad. Sci. USA 81:2864 (1984); Raychaudhuri et al., J. Immunol. 139:271 (1987). Similarly, anti-idiotype vaccines have been developed against infectious organisms, such as Streptococcus pneumoniae (McNamara et al., Science 226:1325 (1984)), hepatitus B virus (Kennedy et al., Science 223:930 (1984)), Escherichia coli K13 (Stein et al., J. Exp. Med. 160:1001 (1984)), Schistosomiasis mansoni (Kresina et al., J. Clin. Invest. 83: 912 (1989)), and Moloney murine sarcoma virus (Powell et al., J. Immunol. 142: 1318 (1989)).

### EXAMPLES

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitations.

Methods of molecular genetics, protein biochemistry, and immunology used but not explicitly described in this disclosure and these Examples are amply reported in the scientific literature and are well within the ability of those skilled in the art.

### Example 1

### Immunization and Specimens

Blood was drawn from 3 healthy volunteers (2 men, 1 woman; ages 24 to 36 years) prior to, 1 and 4 weeks after intramuscular immunization with 23-valent capsular pneumococcal polysaccharide (PPS) vaccine (PNU-IMMUNE®; Lederle-Praxis Biologicals, American Cyanamid Company, Pearl River, N.Y.). Sera were frozen at −20° C. until tested, and peripheral blood mononuclear cells (PBMC) at 1 week were separated by density centrifugation.

### Example 2

### Fusion and Cloning of PPS-Specific Human Monoclonal Antibodies (MAb)

B cells were purified (>95% CD19+ by flow cytometry) by negative selection (StemCell Technologies, Vancouver, Canada). In modifications of published methods (Carroll et al., J Immunol Methods 89:61-72, 1986), fusion of plasma membranes from B cells with the K6H6/B5 heteromyeloma fusion partner (ATCC CRL 1823) at a ratio of 1:2 was achieved with polyethylene glycol (38% w/v PEG×2 min., Sigma Chemical Co., Saint Louis, Mo., #P-7306). The K6H6/B5 heteromyeloma fusion parent is a cross of the NS-1-Ag4 mouse myeloma with a human B cell lymphoma. Carroll et al., J Immunol Methods 89:61-72, 1986. Fused cells were diluted slowly in warm Ca++/Mg++-free PBS (Life Technologies, Inc., Rockville, Md.)×10 minutes, washed, and cultured in selective RPMI 1640 (Life Technologies, Inc.) with 20% fetal bovine serum (FBS, Life Technologies, Inc.) medium containing hypoxanthine, aminopterin and thymidine (HAT, Sigma Chemical Co., #H0262) at 1-3×10⁵ cells/per 200 μL well. Fresh HAT media was added every 3 days for 2-4 weeks, at which time HAT was removed and FBS reduced to 10%. Supernatants of wells were screened by ELISA for IgG, IgM, and IgA antibodies reactive with PPS serotypes 1, 2, 3, 4, 6B, 7F, 8, 9N, 12F, 14, 18C, 19A, 19F, 22F, 23F and 33F (ATCC, Mannassas, Va.) or pneumococcal cell wall polysaccharide (CWPS) exactly as described in Janoff et al., J Clin Invest 104:1139-1147, 1999 and Rubins et al., Infect Immun 67:5979-5984, 1999. These serotypes are those which most commonly cause invasive Streptococcus pneumoniae infections in humans. Cells from wells with detectable antibodies to one or more serotype or cell wall polysaccharide (CPS, Statens Serum Insitiut, Copenhagen, Denmark) were plated in 2-3 96-well plates per original well at 0.6 cells/well on 1×10⁵ washed irradiated (2500 rads) feeder PBMC. Culture supernatants from each well were rescreened for specific antibodies at 2-3 weeks and cells from wells with detectable specific antibodies were replated at 0.6 cells/well. According to Poisson distribution, this approach of cloning reactive cells twice should yield a 99% probability of the final cell expansion being clonal. Yarchoan et al., J Exp Med 157:1-14, 1983; Ukei et al., J Exp Med 171:19-34, 1990; and Lefkovits et al., Immunology Today 5:265-268, 1984. A summary of the functional and physiochemical characterization of 21 S. pneumoniae-specific human monoclonal antibodies obtained by this method is shown in FIG. 1.

### Example 3

### Total and Pneumococcal Polysaccharide-Specific Ig

Levels of total and pneumococcal capsule-specific IgA, IgM, and IgG were measured by enzyme-linked immunosorbent assay (ELISA) as described in Janoff et al., J Chin Invest 104:1139-1147, 1999; Carson et al., J Infect Dis 172:340-345, 1995; and Johnson et al., Infect Immun 64:4339-4344, 1996. Pneumococcal capsule-specific antigens assayed for included both PPS and CPS (Statens SerumInstitut). The heavy chain subclass and light chain utilization by each MAb was determined by ELISA as described (Carson et al., J Infect Dis 172:340-345, 1995) with affinity-purified goat anti-human IgG or IgA as the capture antibodies. Monoclonal antibodies used were as follows: monoclonal mouse anti-human IgA1-biotin (#9030-08, Southern Biotechnology Associates; Birmingham, Ala.), anti-IgA2-biotin (#9140-08; Southern Biotechnology Associates), goat anti-human kappa-HRP (#2060-05; Southern Biotechnology Associates), anti-lambda-HRP (#2070-05; Southern Biotechnology Associates). Monoclonal mouse anti-human IgG1-biotin (Sigma B6775; Sigma Chemical Co., Saint Louis, Mo.) or anti-IgG2-biotin (Sigma B3398) were used as the detector antibodies, with appropriate substrates and developers. Standards included: human myeloma proteins of IgA1 κ (#400109; Calbiochem; San Diego, Calif.), IgA2 λ myeloma (#400110, Calbiochem; San Diego, Calif,), IgG1 κ (#400120; Calbiochem; San Diego, Calif.) IgG2 κ (#400122; Calbiochem; San Diego, Calif.), and IgG3 λ (#400124; Calbiochem; San Diego, Calif.).

The specificity of each MAb was established with a binding and a competitive inhibition assay. Friguet et al., J Immunol Methods 77:305-319, 1985; and Janoff et al., J Immunol 147: 2130-2135, 1991. Increasing concentrations (0.1 to 100 μg/mL) of both homologous and heterologous soluble PPS serotypes, CPS, Haemophilus influenzae type B capsule [poly [3-D-Ribose (1-1) Ribitol-5 phosphate], and tetanus toxoid were incubated overnight at 4° C. with a fixed concentration of monoclonal IgG or IgA reactive with PPS (optical density of 1.0 unit at 410 nm). The ability of these soluble antigens to inhibit binding of the MAb's to solid-phase PPS by ELISA after a 2-hour incubation at 25° C. was compared.

### Example 4

### The specificity or Cross-Reactivity of each MAb was Determined by ELISA

The monoclonal antibodies were tested against 12 pneumococcal polysaccharides (types 1, 2, 3, 4, 6B, 8, 9V, 12F, 14, 19A, 19F, CPS) and six other antigens [thyroglobulin, (Calbiochem, #609312), human double-stranded DNA, (Sigma, #D7011), LPS from Escherichia coli Serotype O127:B8 (Sigma, #L3137), Tetanus Toxoid (W. Latham, Massachusetts Department of Health, Boston), Cholera Toxin from Vibrio cholerae Inaba 569B, (List Biological, #100), and Haemophilus influenzae type B capsule polyribosyl ribitol phosphate (PRP) (Connaught Labs, Swiftwater, Pa.) linked to tyramine hydrochloride (Sigma #T2879)] as ELISA captures. The monoclonal antibodies were applied as samples and binding was detected by affinity-purified goat anti-human IgG or IgA HRPO conjugate (Jackson ImmunoResearch, West Grove, Pa.) and ABTS (Sigma, #A1888) substrate.

### Example 5

### Purification of Polyclonal and Monoclonal PPS-Specific IgA and IgG

As previously described (Janoff et al., J Clin Invest 104:1139-1147, 1999; and Johnson et al., Infect Immun 64:4339-4344, 1996), IgG was purified from serum or culture supernatants by passage over a Protein G Hi-Trap column (Pharmacia Biotech Inc., Piscataway, N.J.). IgM and IgA were separated with affinity columns prepared with goat anti-human IgM or IgA (Southern Biotechnology Associates, Inc., Birmingham, Ala.) coupled to CNBr-activated Sepharose 4B (Pharmacia Biotech Inc.). The purity of all fractions was >98.5% for the specific antibody fractions.

### Example 6

### Physiochemical Characterization of PPS-Specific Antibodies. PPS-Specific Antibodies were Characterized Using the Following Methods

A summary of the physiochemical characterization of 21 S. pneumoniae-specific human monoclonal antibodies is shown in FIG. 1.

a) Antibody Heavy Chain Class

(IgG, IgM, or IgA) and subclass (IgG1, IgG2, IgA1 or IgA2) and light chain usage was determined as described above.

b) Molecular Form of IgA.

The molecular form and proportion of monomeric and polymeric monoclonal antibody was determined by 6% non-denaturing polyacrylamide gel electrophoresis as described in Janoff et al., J Clin Invest 104:1139-1147, 1999; and Johnson et al., Infect Immun 64:4339-4344, 1996. Purified IgA was also fractionated to monomeric and polymeric forms as described in Janoff et al., J Clin Invest 104:1139-1147, 1999; and Johnson et al., Infect Immun 64:4339-4344, 1996 by molecular sieve chromatography using a Sephacryl S-300 HR column (Pharmacia Biotech Inc.) calibrated with polymeric IgA (pIgA) and monomeric (mIgA) standards. The purity of pIgA and mIgA was confirmed by resolution on a 6% continuous non-denaturing polyacrylamide gel stained with Coumassie blue.

c) Antibody avidity.

The avidity of polyclonal and monoclonal antibodies matched for isotype and serotype for pneumococcal capsular polysaccharides was determined as previously described. Rubins et al., J Infect Dis 178:431-440, 1998. The “avidity index” was determined by ELISA as the molar concentration of the mild denaturing agent ammonium thiocyanate required to inhibit 50% of antibody binding to solid phase capsular polysaccharides at sub-maximal concentrations (initial optical density 0.8-1.1 at 405 nm).

d) Antibody VH Gene Sequencing.

As described in Scamurra et al., J Immunol 164:5482-5491, 2000, total cellular RNA was isolated from hybridomas using TRIZOL® Reagent (#15596, Invitrogen Life Technologies, Carlsbad, Calif.) following the manufacturers directions. RNA was DNase I (#1806068-015, Invitrogen Life Technologies) treated and reverse transcribed using MMLV (#28025-013, Invitrogen Life Technologies) to produce cDNA. Sequences of interest were amplified from cDNA using (VH) family specific (VH 1, 3, 4, 5) and IgG and IgA isotype-specific (α, γ) primers for 28 cycles at cycling temperatures and Mg⁺⁺ concentrations determined to be optimal for each primer pair. Primer sequence are described in Scamurra et al., J Immunol 164:5482-5491, 2000. Agarose gel electrophoresis was used to observe presence or absence of product. Amplification products were prepared for sequencing by removing unused primers and dNTPs (#A7170, Wizard PCR Preps DNA Purification System, Promega Corporation, Madison, Wis.; #A7170). Both strands were sequenced in separate reactions with either VH family or CH specific primers at the University of Minnesota Advanced Genetic Analysis Center using BigDye™ (Applied Biosystems Incorporated (ABI), Foster City, Calif.) dideoxy reaction mix and analyzed on an ABI Model 377 Prism DNA sequencer. Sequences were compared with VH sequences contained in two different on-line VH data bases (V BASE: http://www.mrc-cpe.cam.ac.uk/imt-doc, and IMGT: http://imgt.cnusc.fr:8104/). Alignments were performed using DNAPLOT software accessed at these sites. Data were analyzed using the StatView 4.0 statistical program (Abacus Concepts, Berkeley, Calif.). Polynucleotide sequences of the VH region for S. pneumonia-specific human monoclonal antibodies are shown in FIG. 9.

### Example 7

### Functional Characterization of PBS-Specific Antibodies

PPS-specific antibodies were characterized using the following methods.

a) Killing.

The ability of each monoclonal antibody to kill S. pneumoniae isolates matching the capsular specificity of the antibody (e.g. ATCC S. pneumoniae strains 6302, 6303, 6308, and 6326) was tested in a 96-microwell format in modifications of published methods (Gray, Pediatrics 85:694-697, 1990; and Romero-Steiner et al., Clin Diagn Lab Immunol 4:415-422, 1997) as described in Janoff et al., J Clin Invest 104:1139-1147, 1999 and Janoff et al., J Infect Dis 175:975-978, 1997. Log phase Streptococcus pneumoniae was first opsonized for 30 minutes with test Mab or control immune sera, followed by the addition of human neutrophils (1×10⁶) purified from heparinzed blood and 10% baby rabbit complement (#CL3441, Cedarlane Laboratories Limited, Hornby, Ontario). The neutrophils to organism ratio is 400:1. The mixture was incubated at 37° C. for 1 hour with shaking, and lysed in distilled water (1:10); serial dilutions were then plated. The numbers of viable organisms remaining at the end of assay were quantitated and compared to the number from wells containing FBS and complement but no antibody. Percent kill=[(bacteria present with no Ig-bacteria present with IgG or IgA)/bacteria present with no Ig]×100. The killing index was established as the amount of antibody required to effect a 50% reduction in viable organisms (50% kill). A summary of ability of various S. pneumoniae-specific antibodies to kill S. pneumoniae isolates is shown in FIG. 1.

b) Mouse Survival Study.

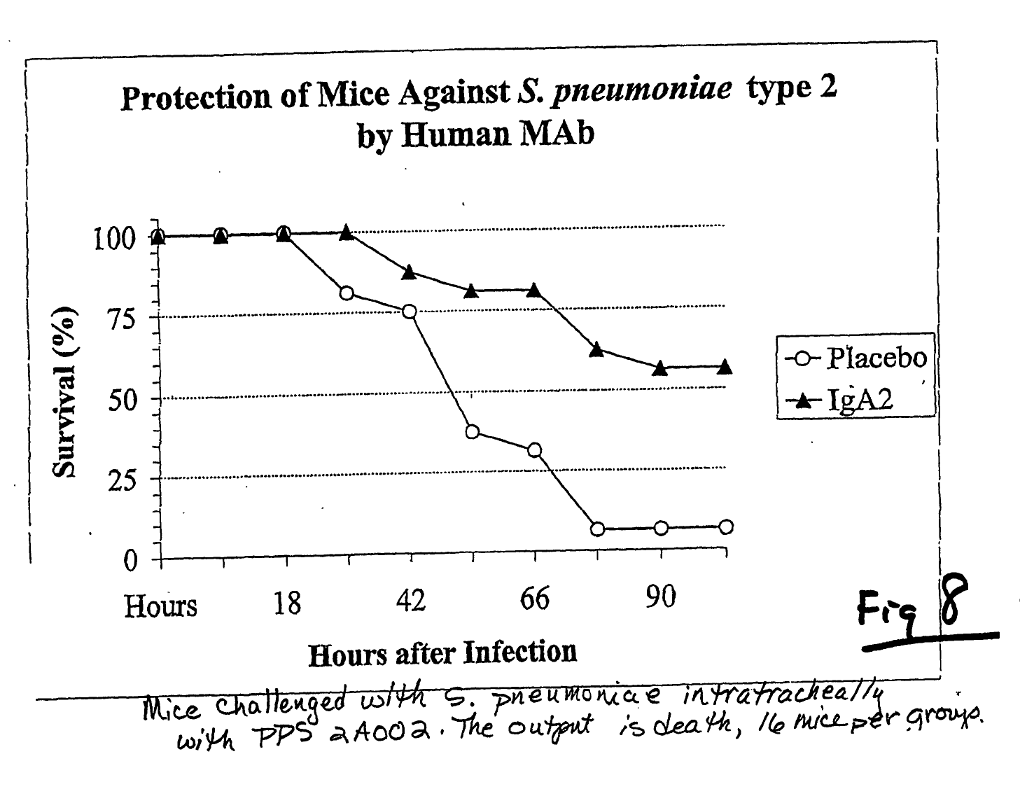

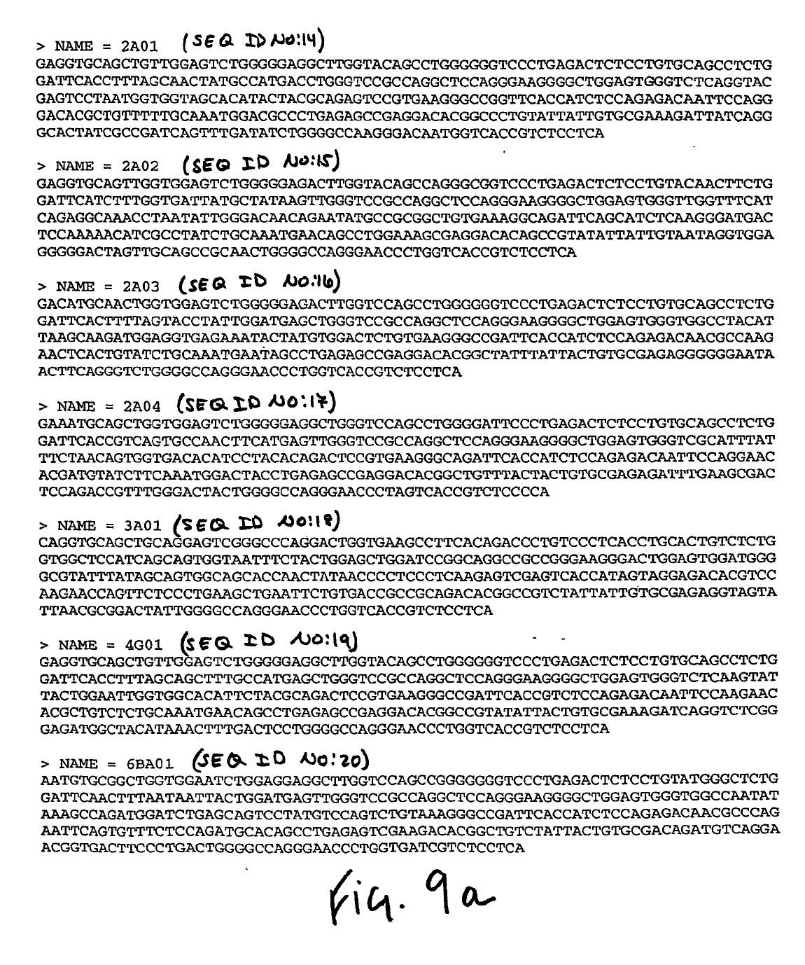

As described (Rubins et al., J Clin Invest 95:142-150, 1995; Rubins et al., Am J Resp Crit Care Med 153:1339-1346, 1996), 32 mice were injected intraperitoneally with 2 μg monoclonal antibody or PBS, then intranasally inoculated with 2×10⁷ S. pneumoniae Serotype 2 (#6302; ATCC, Mannassas, Va.). Survivorship and clinical grades were recorded over a four day period. FIG. 7 shows the virulence of various S. pneumoniae Type 3 isolates in mice. See Neeleman et al., Infect Immun 67(9):4517-24, 1999. Results with the human monoclonal IgA clone 2A02 are shown in FIG. 8.

### Example 8

### Antibody-Dependent Killing of S. pneumoniae by Complement and Phagocytes

As described in Janoff et al., J Clin Invest 104:1139-1147, 1999, purified IgA or control IgG were incubated with shaking for 30 minutes at 25 C. with 1,000 CFU of log-phase type 14 S. pneumoniae (American Type Culture Collection 6314, Manassas, Va., USA). Phagocytes (4×10⁵) and a specified human or rabbit serum complement source (10%) were added, incubated at 37° C. for 1 hour with shaking, and lysed in distilled water (1:10); serial dilutions were then plated. Phagocytes were either freshly isolated peripheral blood neutrophils (PMN) or cultured HL-60 cells (Certified Cell Line 240; American Type Culture Collection) treated with 120 mM dimethyl formamide to induce differentiation. The absence of IgG and IgA bound to purified human neutrophils was verified by FACS® with affinity-purified FITC-labeled goat F(ab′)₂ anti-human Fcγ and Fcα (Southern Biotechnology Associates), respectively. Cells were also washed 3 times to remove soluble Igs from the donor. To determine the antibody specificity, killing assays were performed with and without preincubation of purified IgG and IgA with 20 g/mL cell wall polysaccharide and type 14 PPS; control wells contained 10% heat-inactivated FCS (GIBCO BRL, Grand Island, N.Y., USA). Killing activity of antibodies with complement and phagocytes (or with activated phagocytes and no complement) was calculated using the following formula: percent kill=[(bacteria present with no Ig bacteria present with IgG or IgA)/bacteria present with no Ig]×100. To determine the receptors involved in killing, selected experiments were performed with preincubation of phagocytes with isotype controls (Southern Biotechnology Associates and PharMingen, San Diego, Calif., USA) and unlabeled antibodies with blocking activity against complement receptor CR1 (CD35; DAKO Corp.), CR3 (CD11b; BioSource International, Camarillo, Calif., USA), Fcα receptor (CD89; Medarex Inc., Annandale, N.J.), Fcγ receptor I (CD64; PharMingen), Fcγ receptor II, and Fcγ receptor III (CD32 and CD16; Medarex Inc.).

### Example 9

### Analysis of Uptake of S. pneumoniae by Phagocytes

As described in Janoff et al., J Clin Invest 104:1139-1147, 1999, specified concentrations of antibody (purified IgA or IgG) and S. pneumoniae (2×10⁵ CFU) were combined in a total volume of 50 L and shaken in Immulon I wells (Dynex Technologies, Chantilly, Va., USA) for 30 minutes at 25° C. Baby rabbit complement and purified neutrophils (2×10⁵ per well) were added, and the final mixture (100 μL) was incubated at 37° C. for 1 hour with shaking. Well contents were either removed for cytospin (Cytospin 2, Shandon Inc., Pittsburgh, Pa., USA) or slow spun at 150 g to pellet neutrophils. Cytospins were stained with Wright-Giemsa for light microscopy. Cell pellets were fixed in 2.5% glutaraldehyde and processed for transmission electron microscopy as described. Whole complement was excluded and neutrophils were preactivated with 10⁻⁸ M recombinant human TNF-α (R&D Systems Inc., Minneapolis, Minn., USA) or 10⁻⁹ M purified C5a (CalBiochem-Novabiochem Corp., San Diego, Calif., USA), or both agents together.

### Example 10

### Characterization of a Panel of Human Monoclonal Antibodies Specific for Capsular Polysaccharide Antigen of S. pneumoniae

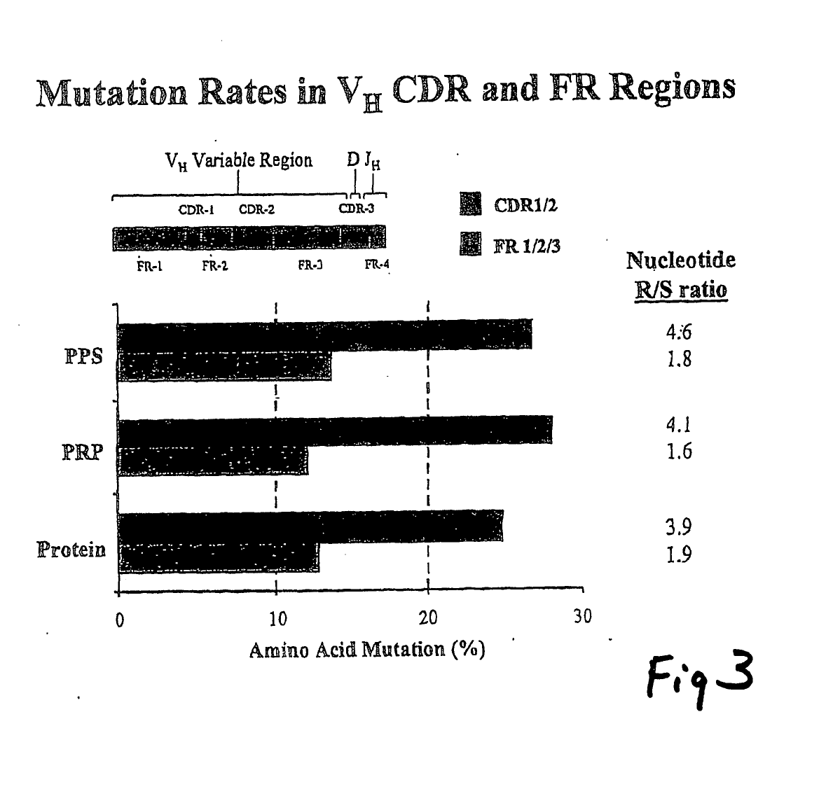

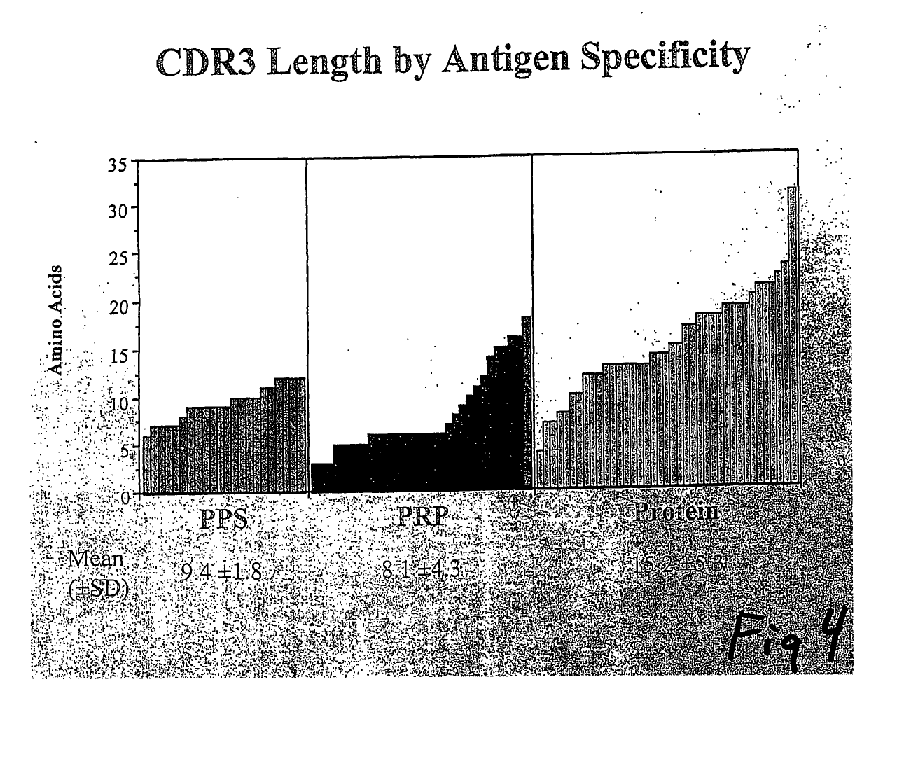

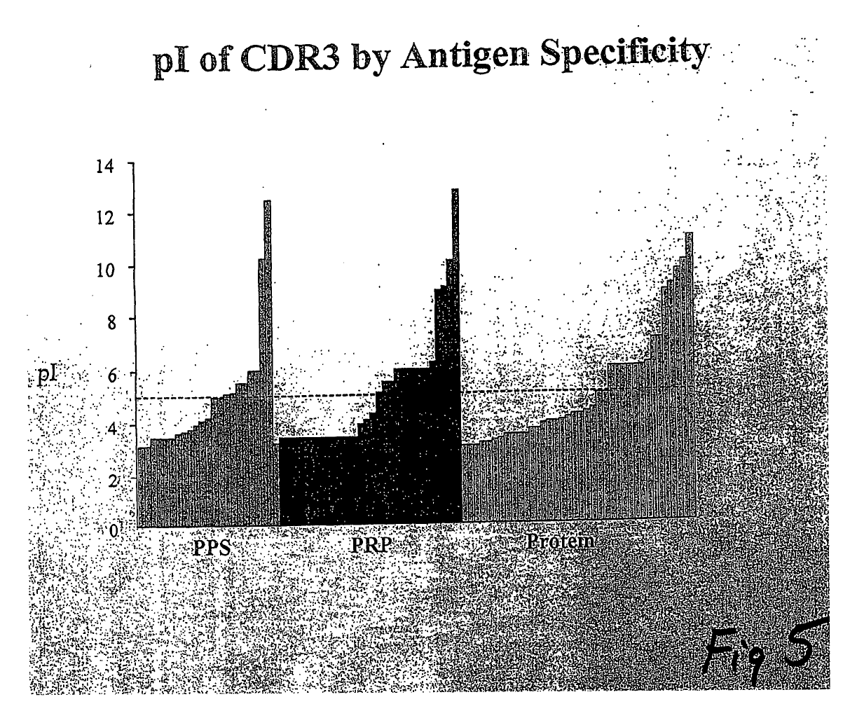

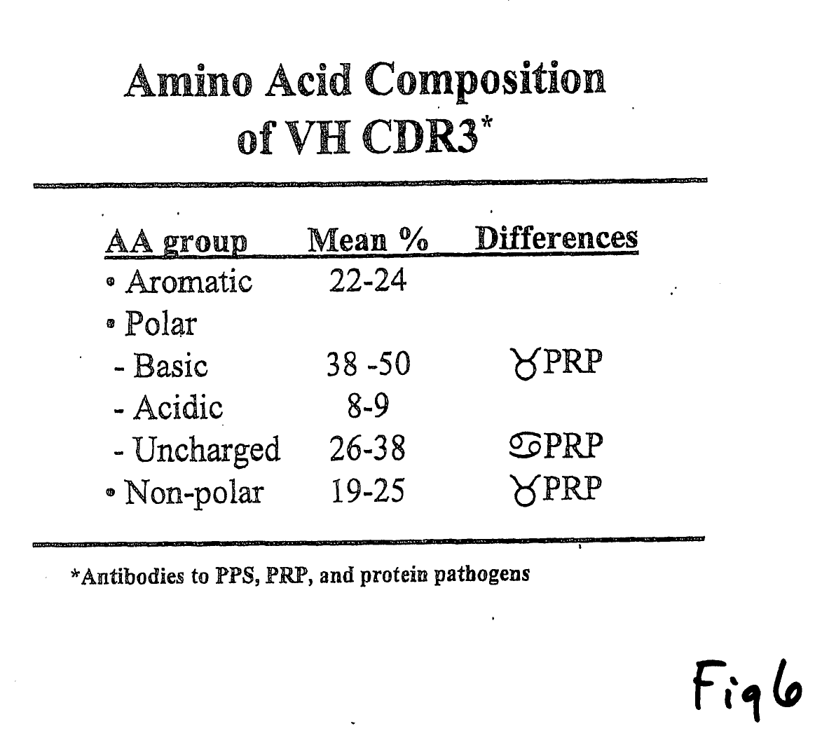

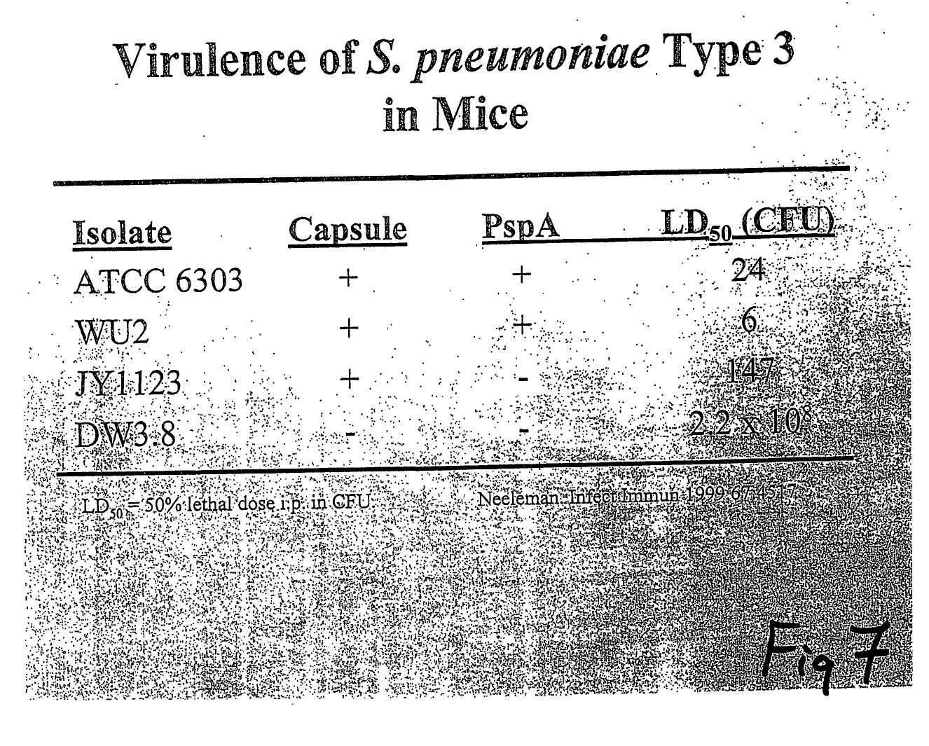

As outlined in Example 2, a panel of S. pneumoniae-specific human monoclonal antibodies was generated and characterized. The ability of various S. pneumoniae-specific antibodies to kill S. pneumoniae isolates was determined by the method outlined in Example 7(a). Eleven monoclonal antibodies (910 IgA and 2 IgG) reactive with PPS serotypes 1, 2, 3, 6B, and 8 were tested. Five of the eleven antibodies tested supported killing of S. pneumoniae in the presence of complement and phagocytes (LD50=20-200 ng/mL of IgG or IgA). See FIG. 1. Avidity indexes, which reflect the strength of binding of the antibody to PPS, ranged broadly from 0.5 to 1.6, but were not directly related to killing activity. Each IgA monoclonal antibody was produced in both monomeric and polymeric configurations, but the latter killed more efficiently. Molecular characterization of the antigen-binding variable region of heavy chain genes (VH) of the human monoclonal antibodies shown in FIG. 1 and thirteen additional antibodies from the literature revealed a striking predominance of monoclonal antibodies utilizing genes of the prominent VH3 family, esp. V3-15 and V3-23. See FIG. 2. During affinity maturation, mutations are introduced into antibodies carrying unmutated germline VH genes and those with increased affinity for antigen and presumably enhanced function are selected. As shown in FIG. 3, rates of amino acid mutations in the hypervariable antigen-associated CDR1/2 regions were similar in monoclonal antibodies reactive with PPS compared with those in 29 monoclonal antibodies reactive with the polysaccharide capsule of Haemophilus influenzae type b (Hib) and 38 monoclonal antibodies specific for pathogen-derived protein antigens (24-28%). The replacement to silent (R/S) ratios, which reflect antigen-driven selection of antibodies were also similar in CDR1/2 (PPS 4.6; Protein 3.9; Hib 4.1). As shown in FIG. 4, among PPS monoclonal antibodies, mean CDR3 lengths were shorter (9.6±0.4 aa) compared with those in monoclonal antibodies to proteins (14.8±1.6 aa; P=0.002), and similar to those to Hib (8.2±8 aa), suggesting differences in selection among these antibodies for effective antigen recognition. In summary, a bank of PPS-specific human monoclonal antibodies with the ability to support killing of the organism was developed. These monoclonal antibodies utilize a limited range of VH genes, primarily showing usage of a VH3 gene segment, demonstrate isotype class switching, high rates of mutation, and shorter CDR3 lengths than in those reactive with protein antigens.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

### Sequence Listing Free Text

SEQ ID NOS:1-13 Amino acid sequence of the D/J CDR3 region of various S. pneumoniae-specific human monoclonal antibodies.

SEQ ID NOS:14-26 Polynucleotide sequences of the V_{H} region of various S. pneumoniae-specific human monoclonal antibodies.

What is claimed is:

1. A human monoclonal antibody that specifically binds to a capsular or cell wall-associated polysaccharide antigen of Streptococcus pneumoniae, said human monoclonal antibody selected from the group consisting of 1A01, 2A01, 2A02, 2A03, 2G01, 2A04, 3A01, 3G01, 4G01, 7A01, 8A01, 8A04, 8A02, 8G01, 9G01, 33G01, 18CA01, 22FG01, 6BG01, 6BA01 and CPSM01; and antigen-binding fragments thereof.

2. The antigen-binding fragment of claim 1, wherein said antigen-binding fragment is a scFv, a Fv, a Fab′, a Fab, a diabody, a linear antibody or a F(ab′)₂.

3. An antibody or antigen-binding fragment that specifically binds to a capsular or cell wall-associated polysaccharide antigen of Streptococcus pneumoniae, said antibody or antigen-binding fragment comprising a VH region amino acid sequence encoded by a polynucleotide sequence selected from the group consisting of SEQ ID NO:14-26.

4. An antibody or antigen-binding fragment that specifically binds to a capsular or cell wall-associated polysaccharide antigen of Streptococcus pneumoniae, said antibody or antigen-binding fragment comprising a CDR3 amino acid sequence selected from the group consisting of SEQ ID NO:1-13.

5. A human monoclonal antibody that specifically binds to a capsular or cell wall-associated polysaccharide antigen of Streptococcus pneumoniae, said human monoclonal antibody secreted by a hybridoma cell line selected from the group consisting of Z727 1D8 1C7 2B8; BBK040 1F2 1G7 2G8; Z531 3D7 5A9 1A9; Z531 4B6 3F5 1E2; Z727 1C11 1E12 3B10; Z727 1D11 1D11 1F6; Z531 1F9 3H9 1B7; Z727 2G6 1B5 3B7; Z727 2B5; Z727 1G8 1B10 3E1; 1A8 1A3/1F7/1H59; BBK038 2F4 F11 1B6; BBK040 1D9 2B5; BBK040 4E4 2D1 1A5; BBK038 1B8 E2 1F6; Z727 1G8 1B10 1E12/3D5; Z727 1C11 1F7 2A8; Z727 1G8 1B3 1B11/1F9; Z727 1D11 1H6; BBK040 4E4 4A2; Z727 1D11 1E7 2E1; and BBK041 2G5 1G2.

6. A hybridoma cell line selected from the group consisting of Z727 1D8 1C7 2B8; BBK040 1F2 1G7 2G8; Z531 3D7 5A9 1A9; Z531 4B6 3F5 1E2; Z727 1C11 1E12 3B10; Z727 1D11 1D11 1F6; Z531 1F9 3H9 1B7; Z727 2G6 1B5 3B7; Z727 2B5; Z727 1G8 1B10 3E1; 1A8 1A3/1F7/1H59; BBK038 2F4 F11 1B6; BBK040 1D9 2B5; BBK040 4E4 2D1 1A5; BBK038 1B8 E2 1F6; Z727 1G8 1B10 1E12/3D5; Z727 1C11 1F7 2A8; Z727 1G8 1B3 1B11/1F9; Z727 1D11 1H6; BBK040 4E4 1A2; Z727 1D11 1E7 2E1; and BBK041 2G5 1G2.

7. A pharmaceutical composition comprising at least one of the antibodies of claim 1, or antigen-binding fragments thereof.

8. A pharmaceutical composition comprising at least one of the antibodies of claim 1, or antigen-binding fragments thereof, and a pharmaceutically acceptable carrier.

9. A pharmaceutical composition comprising a cocktail, said cocktail comprising at least one of the human monoclonal antibodies of claim 1, or antigen-binding fragments thereof.

10. A method for treating an individual with a Streptococcus pneumoniae infection comprising passively administering a therapeutically effective amount of a pharmaceutical composition according to claim 7 to said individual.

11. The method of claim 10, wherein said individual is a human patient.

12. The method of claim 11, wherein said human patient is suffering from a condition selected from the group consisting of pneumococcal pneumonia, meningitis, otitis media, sinusitis, sickle cell anemia, hypogammaglobulinemia, asplenia and bacteremia.

13. The method of claim 11, wherein said patient has an impaired ability to produce antibodies.

14. A method for preventing a Streptococcus pneumoniae infection in an individual comprising passively administering a prophylactically effective amount of a pharmaceutical composition according to claim 7 to said individual.

15. The method of claim 14 wherein said patient suffers from a condition selected form the group consisting of HIV/AIDS, functional or surgical asplenia, chronic lymphocytic leukemia (CLL), multiple myeloma (MM), hypogammaglobulinemia and sickle cell anemia.

16. The method of claim 11, wherein said patient is under two years of age or experiences recurrent meningitis because of a skull fracture or other structural defect.

17. The method of claim 14, wherein said patient is under two years of age or experiences recurrent meningitis because of a skull fracture or other structural defect.

18. A method of detecting Streptococcus pneumoniae comprising contacting a biological sample with an antibody of claim 1.

19. A standard for use in a diagnostic immunoassay, said standard comprising at least one of the antibodies of claim 1.

20. A method for treating an individual with a Streptococcus pneumoniae infection comprising:
a) selecting at least one human monoclonal antibody that specifically binds to a capsular or cell wall-associated polysaccharide antigen of Streptococcus pneumoniae, utilizes a heavy-chain immunoglobulin gene from the VH3 gene family, comprises a shortened CDR3 region of 8-12 amino acids, demonstrates an increased mutation rate of 6.5±0.9% and demonstrates a decreased proportion of solvent-exposed amino acids relative to an antibody specific for a protein antigen; and
b) passively administering a therapeutically effective amount of said at least one human monoclonal antibody to said individual.

21. A method for preventing a Streptococcus pneumoniae infection in an individual comprising:
a) selecting at least one human monoclonal antibody that specifically binds to a capsular or cell wall-associated polysaccharide antigen of Streptococcus pneumoniae, utilizes a heavy-chain immunoglobulin gene from the VH3 gene family, comprises a shortened CDR3 region of 8-12 amino acids, demonstrates an increased mutation rate of 6.5±0.9% and demonstrates a decreased proportion of solvent-exposed amino acids relative to an antibody specific for a protein antigen; and
b) passively administering a prophylactically effective amount of said at least one human monoclonal antibody to said individual.

22. An anti-idiotypic antibody that specifically binds to an antibody of claim 1.
